(19) United States
(12) Patent Application Publication
(10) Pub. No.: US 2006/0128744 A1
(43) Pub. Date: Jun. 15, 2006

(54) Use of 5-HT6 agonist for the treatment and prevention of neurodegenerative disorders

(75) Inventors: Lee Erwin Schechter, Toms River, NJ (US); Kevin Pong, Robbinsville, NJ (US); Margaret Maria Zaleska, Narberth, PA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/301,445

(22) Filed: Dec. 13, 2005

Related U.S. Application Data
(60) Provisional application No. 60/635,766, filed on Dec. 14, 2004.

(51) Int. Cl. A61K 31/4745; A61K 31/404

(52) U.S. Cl. 514/300; 514/418

(57) ABSTRACT

The present invention provides method for the treatment, amelioration or prevention of a neurodegenerative disorder in a patient in need thereof which comprises administering to said patient an effective amount of a 5-hydroxytryptamine-6 agonist.

This application claims the benefit under 35 U.S.C. §119(e) to co-pending U.S. provisional application No. 60/635,766, filed Dec. 14, 2004, which is hereby incorporated by reference in its entirety.

### BACKGROUND OF THE INVENTION

Glutamate is the predominant neurotransmitter in the central nervous system and plays and important role in neuroplasticity. Excessive extracellular levels of glutamate have been associated with the pathophysiology of both acute neurodegenerative disorders such as stroke, transient ischemic attack or spinal/brain trauma, and chronic neurodegenerative disorders such as epilepsy, Alzheimer's Disease, amyotrophic lateral sclerosis, Huntington's disease, Parkinson's Disease, AIDS dementia and retinal diseases¹. Compounds which inhibit the release of glutamate would be expected to be useful in the treatment of chronic diseases in which glutamate dysfunction plays a role, such as chronic neurodegeneration, Alzheimer's Disease, Huntington's Disease, Parkinson's Disease, amyotrophic lateral slerosis epilepsy, schizophrenia, AIDS dementia, or retinal diseases. Further, compounds which inhibit or attenuate the release of glutamate may also provide potential neuroprotective agents for the treatment of ischemia resulting from stroke, transient ischemic attack or brain//spinal trauma² or of ischemia resulting from surgery where the blood flow must be halted for a period of time (for example, cardiac by-pass surgery)³. Approximately 5-6 million people, in America alone, are afflicted with chronic or acute neurodegenerative disorders. Accordingly there is a need for an effective compound to treat and prevent neurodegenerative conditions.

Therefore it is an object of the present invention to provide a method for the treatment or prevention of neurodegenerative disorders.

It is another object of this invention to provide a source of neuroprotective agents.

Further objects and features of the invention will become more apparent by the detailed description set forth hereinbelow.

### BIBLIOGRAPHY

¹ Holt, W. F. et al, Glutamate in Health and Disease: The Role of Inhibitors, Neuroprotection in CNS Diseases, Bar, P. R. and Beal, M. F., ed., Marcel Dekker, Inc., New York, 1997, pp 87-199.
Engelsen, B. A. et al, Alterations in Excitatory Amino Acid Transmitters in Human Neurological Disease and Neuropathology, Neurotoxicity of Excitatory Amino Acids, Guidotti, A., ed., Raven Press Ltd., New York, 1990, pp. 311-332.
Ince, P. G., et al, The Role of Excitotoxicity in Neurological Disease, Res. Contemp. Pharmacother, (1997), 8, pp. 195-212.
Meldrum, B. S., The Gutamate Synapse as a Therapeutical Target: Perspective for the Future, Prog. Brain Res., (1998), pp. 441-458.
²Koroshetz, W. J. and Moskowitz, M. A., Emerging Treatment for Stroke in Humans, Trends in Pharmacol. Science, (1996), 17, pp. 227-233.
Dunn, C. D. R., Stroke: Trends, Tratments and Markets, Scrip Reports, PJB Publications, Richmond Va., 1995.
³Arrowsmith, J. E., et al, Neuroprotection of the Brain During Cardiopulmonary Bypass: A Randomized Trial of Remacemide During Coronary Artery Bypass in 171 Patients, Stroke, (1998), 29, pp. 2357-2362.

### DESCRIPTION OF DRAWINGS

FIG. 1. FIG. 1 is a schematic representation of the neuroprotective effect of a 5-HT6 agonist (Test Compound B) on neuronal survival, as determined by a neurofilament ELISA.

FIG. 2. FIG. 2 is a schematic representation of the neuroprotective effect of a 5-HT6 agonist (Test Compound B) on neurite outgrowth wherein the data are expressed as total neurite length.

FIG. 3. FIG. 3 is a schematic representation of the neuroprotective effect of a 5-HT6 agonist (Test Compound C) against OGD-induced neuronal cell death in cerebellar granule neurons.

FIG. 4. FIG. 4 is a schematic representation of the neuroprotective effect of a 5-HT6 agonist (Test Compound C) against Potassium withdrawal-induced apoptosis in cerebellar granule neurons.

FIG. 5. FIG. 5 is a schematic representation of the effect of a 5-HT6 agonist (Test Compound B) on the brain derived neurotrophic factor (BDNF) protein level in cultured cortical neurons.

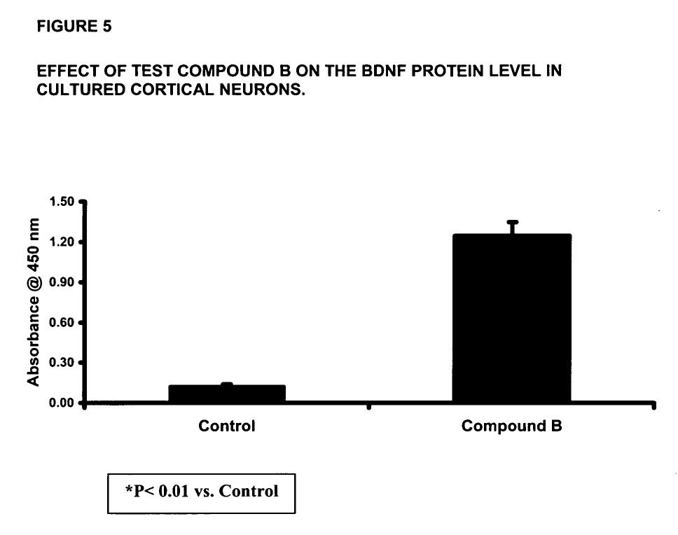

### SUMMARY OF THE INVENTION

The present invention provides a method for the treatment of a neurodegenerative disorder in a patient in need thereof which comprises providing to said patient a therapeutically effective amount of a 5-hydroxytryptamine-6 agonist.

Also provided is a pharmaceutical composition, for use in the treatment of a neurodegenerative disorder, comprising a pharmaceutically acceptable carrier and an effective amount of a 5-hydroxytryptamine-6 agonist.

### DETAILED DESCRIPTION OF THE INVENTION

Dysfunctional glutamate release, and in particular excessive glutamate release, is associated with the pathohysiology of both acute neurodegenerative disorders such as stroke, transient ischemic attack or spinal/brain trauma, and chronic neurodegenerative disorders such as epilepsy, Alzheimer's Disease, amyotrophic lateral sclerosis, Huntington's disease, Parkinson's Disease, AIDS dementia or retinal diseases.

Additionally, endogenous GABA function appears to be markedly decreased in the brain following ischemic brain injury (Green A. R., et al., Neuroscience Letters, 1992, 138, 141-144; and Green A. R., et al., Neuropharmacology, 2000, 39, 1483-1493). Experimental studies demonstrate that a drug capable of stimulating GABAergic function (eg., GABA agonist) when combined with an agent capable of decreasing glutamatergic neurotransmission (eg., glutamate antagonist) may have neuroprotective effects (Lyden et al., Journal of Neurotrauma, 1995, 12(2), 223-230.

Moreover, brain derived neurotrophic factor (BDNF), a member of the nerve growth factor family of proteins, has been shown to promote neuroprotection and neuroregeneration of neurons. (Binder, D. K. and Scharfman, H. E., Growth Factors, 2004, 22(3), pp. 123-131) Compounds which increase the level of BDNF may thereby promote the survival and plasticity of neurons and comensurately demonstrate neuroprotective effects. (Nagappan, G. and Lu, B., Trends in Neurosciences, 2005, 28(9), pp. 464-471).

Surprisingly, it has now been found that a 5-HT6 receptor agonist effectively increases extracellular GABA concentrations and reduces glutamate release caused by ischemic-inducing agents. Further, it has now been found that a 5-HT6 agonist effectively increases the level of brain derived neurotrophic factor (BDNF) protein in cultured cortical neurons. These findings strongly indicate that a 5-HT6 agonist has neuroprotective properties, including promoting the survival and plasticity of neurons, and may be an effective therapeutic for the treatment and prevention of neurodegenerative disorders.

Advantageously, the use of a selective 5-HT6 agonist for the treatment of neurodegenerative disorders may have minimal side effects. Due to the exclusive localization of the 5-HT6 receptor in the brain, peripheral organ systems, such as the cardiovascular system, would not be affected by a 5-HT6 agonist. Further, the specificity of the 5-HT6 agonist may lead to acute onset of action and enhanced therapeutic efficacy.

A 5-HT6 agonist is defined herein as any compound which is capable of binding with the 5-HT6 receptor, as determined by conventional binding assay methods well known in the art, and which demonstrates a 25% or greater, preferably 50% or greater, more preferably 70% or greater, particularly 90% or greater, accumulation of adenosine 3′5′-cyclic monophophate (cAMP) at the 5-HT6 receptor site, as compared to serotonin.

Among the 5-HT6 agonists suitable for use in the method of the invention are those compounds described in WO 99/47516, GB 2,341,549, U.S. Pat. No. 6,770,642, U.S. Pat. No. 6,767,912, U.S. Pat. No. 6,800,640, U.S. Pat. No. 6,727,246, and US 2003-0236278. U.S. Pat. No. 6,770,642, U.S. Pat. No. 6,767,912, U.S. Pat. No. 6,800,640, U.S. Pat. No. 6,727,246, and US 2003-0236278 are incorporated herein by reference thereto.

Methods to prepare 5-HT6 agonists suitable for use in the method of invention are described in the above-mentioned patents and patent applications and also in U.S. Pat. No. 4,940,710.

Preferred 5-HT6 agonists suitable for use in the method for the invention include those compounds disclosed in WO 99/147516, GB 2,341,549, U.S. Pat. No. 6,770,642 and US 2003-0236278 and having the structure of formula I 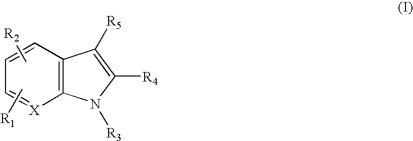
wherein
X is CH or N;
R₁ and R₂ are each independently H, halogen, CN, OCO₂R₁₂, CO₂R₁₃, CONR₁₄R₁₅, CNR₁₆NR₁₇R₁₈, SOₘR₁₉, NR₂₀R₂₁, OR₂₂, COR₂₃ or a C₁-C₆alkyl, C₂-C₆alkenyl, C₂-C₆alkynyl, C₃-C₆cycloalkyl, cycloheteroalkyl, aryl or heteroaryl group each optionally substituted;
R₃ is SO₂R₈ when X is CH or (CH₂)ₙNR₆R₇ when X is N;
R₄ is H, halogen, or a C₁-C₆alkyl, C₁-C₆alkoxy, aryl or heteroaryl group each optionally substituted;
R₅ is (CH₂)ₙNR₆R₇ when X is CH or SO₂R₈ when X is N;
n is an integer of 2 or 3;
R₆ and R₇ are each independently H or a C₁-C₆alkyl, C₂-C₆alkenyl, C₂-C₆alkynyl, C₃-C₆cycloalkyl, cycloheteroalkyl, aryl or heteroaryl group each optionally substituted, or R₆ and R₇ may be taken together with the atom to which they are attached to form an optionally substituted 5- to 7-membered ring optionally containing an additional heteroatom selected from O, N or S;
R₈ is an optionally substituted aryl, heteroaryl or 8- to 13-membered bicyclic or tricyclic ring system having a N atom at the bridgehead and optionally containing 1, 2 or 3 additional heteroatoms selected from N, O or S;
m is 0 or an integer of 1 or 2;
R₁₂, R₁₃, R₁₉ and R₂₃ are each independently H or a C₁-C₆alkyl, C₂-C₆alkenyl, C₂-C₆alkynyl, C₃-C₆cycloalkyl, cycloheteroalkyl, aryl or heteroaryl group each optionally substituted;
R₁₄, R₁₅ and R₂₂ are each independently H or an optionally substituted C₁-C₆alkyl group; and
R₁₆, R₁₇, R₁₈, R₂₀ and R₂₁ are each independently H or an optionally substituted C₁-C₄alkyl group; or R₂₀ and R₂₁ may be taken together with the atom to which they are attached to form a 5- to 7-membered ring optionally containing another heteroatom selected from O, N or S; or
   the stereoisomers thereof or the pharmaceutically acceptable salts thereof.

As used in the specification and claims, the term halogen designates F, Cl, Br or I and the term cycloheteroalkyl designates a 5-7 membered ring system containing 1 or 2 heteroatoms, which may be the same or different, selected from nitrogen, oxygen and sulfur and optionally containing one double bond. Exemplary of the cycloheteroalkyl ring systems included in the term as designated herein are the following rings wherein X₁ is NR, O or S; and R is H or an optional substituent as described hereinbelow:

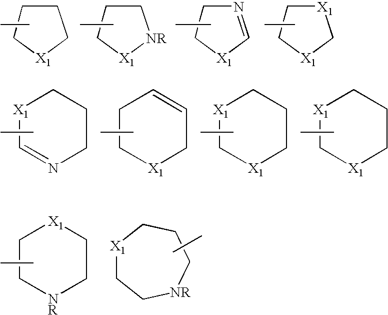

Similarly, as used in the specification and claims, the term heteroaryl designates a five to ten membered aromatic ring system containing 1, 2 or 3 heteroatoms, which may be the same or different, selected from N, O or S. Such heteroaryl ring systems include pyrrolyl, azolyl, oxazolyl, thiazolyl, imidazolyl, furyl, thienyl, quinolinyl, isoquinolinyl, indolinyl, benzothienyl, benzofuranyl, benzisoxazolyl or the like.

The term aryl designates a carbocyclic aromatic ring system, e.g., having 6-14 carbon atoms such as phenyl, naphthyl, anthracenyl or the like.

The term haloalkyl as used herein designates a CₙH₂ₙ₊₁ group having from one to 2n+1 halogen atoms which may be the same or different and the term haloalkoxy as used herein designates an OCₙH₂ₙ₊₁ group having from one to 2n+1 halogen atoms which may be the same or different.

Exemplary of the 8- to 13-membered bicyclic or tricyclic ring systems having a N atom at the bridgehead and optionally containing 1, 2 or 3 additional heteroatoms selected from N, O or S included in the term as designated herein are the following ring systems wherein W₂ is NR, O or S; and R is H or an optional substituent as described hereinbelow:

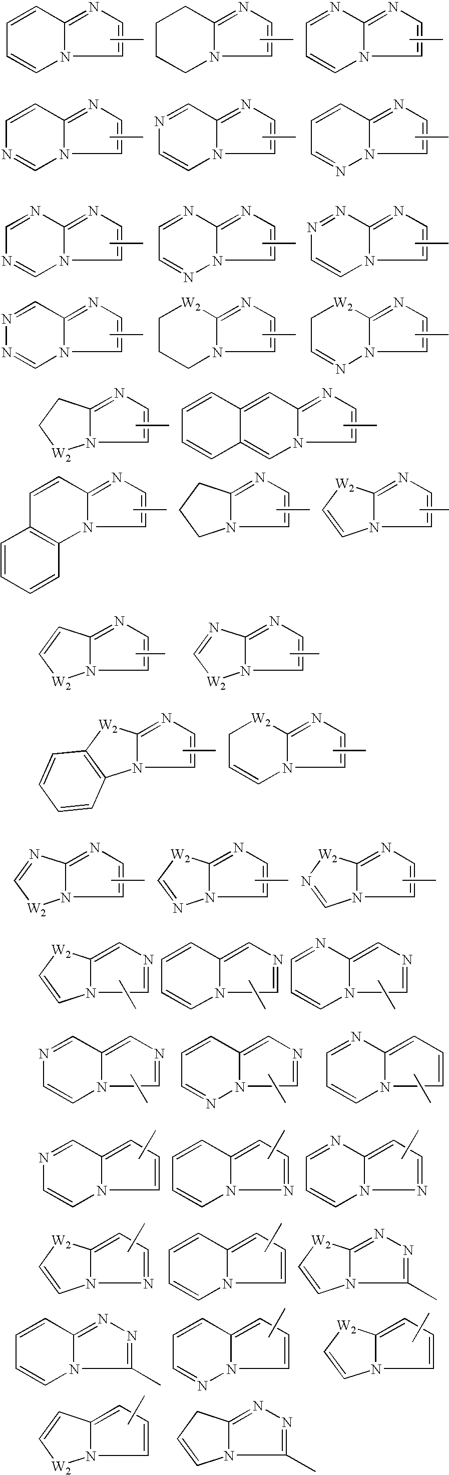

In the specification and claims, when terms such as C₁-C₆alkyl, C₂-C₆alkenyl, C₂-C₆alkynyl, C₃-C₇cycloalkyl, cycloheteroalkyl, aryl or heteroaryl are designated as being optionally substituted, the substituent groups which are optionally present may be one or more, e.g., 2 or 3, of those customarily employed in the development of pharmaceutical compounds or the modification of such compounds to influence their structure/activity, persistence, absorption, stability or other beneficial property. Specific examples of such substituents include halogen atoms, nitro, cyano, hydroxyl, alkyl, haloalkyl, alkoxy, haloalkoxy, amino, alkylamino, dialkylamino, formyl, alkoxycarbonyl, carboxyl, alkanoyl, alkylthio, alkylsuphinyl, alkylsulphonyl, carbamoyl, alkylamido, phenyl, phenoxy, benzyl, benzyloxy, heterocyclyl (such as heteroaryl or cycloheteroalkyl) or cycloalkyl groups, preferably halogen atoms or lower alkyl groups. Typically, 0-3 substituents may be present. When any of the foregoing substituents represents or contains an alkyl substituent as a group or part of a group, this may be linear or branched and may contain up to 12, preferably up to 6, more preferably up to 4 carbon atoms.

Compounds of formula I may be prepared according to the methods described in GB 2,341,549, U.S. Pat. No. 6,770,642 and US 2003-0236278.

More preferred 5-HT6 agonists suitable for use in the method of invention are 1-sulfonyltryptamine derivatives including those compounds of formula I wherein X is CH; n is 2; and R₈ is a phenyl or imidazo[2,1-b][1,3]thiazolyl group each optionally substituted. Another group of more preferred 5-HT6 agonists suitable for use in the inventive method are 3-sulfonylazaindole derivatives including those compounds of formula I wherein X is N; n is 2; and R₈ is a phenyl or imidazo[2,1-b][1,3]thiazolyl group each optionally substituted.

Among the 5-HT6 agonist compounds of formula I suitable for use in the method of invention are: 2-{1-[6-chloroimidazo[2,1-b][1,3]thiazol-5-yl)sulfonyl]-1H-indol-3-yl}ethanamine; (2-{3-[(2,5-dimethoxyphenyl)sulfonyl]-1H-pyrrolo[2,3-b]pyridin-1-yl}ethyl)amine; N-(2-{3-[(3-fluorophenyl)sulfonyl]-1H-pyrrolo[2,3-b]pyridin-1-yl}ethyl)-N,N-dimethylamine; 2-{[1-(phenylsulfonyl)-1H-indol-3-yl]ethyl}-N,N-dimethylamine; the pharmaceutically acceptable salts thereof; or the stereoisomers thereof.

Compounds which exhibit 5-HT6 receptor agonist activity may form acid addition salts with acids, such as conventional pharmaceutically acceptable acids, for example, acetic, phosphoric, sulfuric, hydrochloric, hydrobromic, citric, maleic, malonic, mandelic, succinic, fumaric, acetic, lactic, tartaric, salicylic, nitric, sulfonic, p-toluene, sulfonic, methane sulfonic acid or the like. Salts of 5-HT6 receptor agonists are therefore embraced by the method of the invention.

The method of the invention includes esters, carbamates or other conventional prodrug forms of a 5-HT6 agonist compound, which in general, are functional derivatives of the 5-HT6 agonist compounds and which are readily converted to the active moiety in vivo. Correspondingly, the method of the invention embraces the treatment of a neurodegenerative disorder with a 5-HT6 agonist, such as a compound of formula I or with a compound which is not specifically disclosed but which, upon administration, converts to a 5-HT6 agonist in vivo. Also included are metabolites of the 5-HT6 agonist compounds defined as active species produced upon introduction of said agonists into a biological system.

Compounds which exhibit 5-HT6 receptor agonist activity may exist as one or more stereoisomers. The various stereoisomers include enantiomers, diastereomers, atropisomers and geometric isomers. One skilled in the art will appreciate that one stereoisomer may be more active or may exhibit beneficial effects when enriched relative to the other stereoisomer(s) or when separated from the other stereoisomer(s). Additionally, the skilled artisan knows how to separate, enrich or selectively prepare said stereoisomers. Accordingly, the method of invention embraces 5-HT6 agonist compounds, the stereoisomers thereof and the pharmaceutically acceptable salts thereof. Said agonist compounds may be present as a mixture of stereoisomers, individual stereoisomers, or as an optically active or enantiomerically pure form.

Accordingly, the present invention provides an effective method for the treatment and prevention of neurodegenerative disorders in a patient in need thereof which comprises providing to said patient a therapeutically effective amount of a 5-HT6 agonist as described hereinabove.

In one embodiment of the invention there is provided a method for increasing brain-derived neurotrophic factor protein in a patient in need thereof which comprises providing to said patient a therapeutically effective amount of a 5-HT6 agonist as described hereinabove.

Said 5-HT6 agonist may be provided by oral or parenteral administration or by any common manner known to be an effectual administration of a therapeutic agent to a patient in need thereof.

A therapeutically effective amount, as used herein, is an amount sufficient to provide a degree of neuroprotection, or to treat, prevent or ameliorate the symptoms associated with neurodegeneration or excessive or dysfunctional glutamate release.

Neurodegenerative disorders suitable for treatment by the method of the invention include both chronic neurodegenrative disorders and acute neurodegenerative disorders. Chronic neurodegenerative disorders include, but are not limited to, Alzheimer's Disease, amyotrophic lateral sclerosis, Huntington's disease, Parkinson's Disease, AIDS dementia, epilepsy or retinal diseases. Acute neurodegenerative disorders include, but are not limited to, stroke, head or spinal trauma, or asphyxia. Stroke includes acute thromboembolic stroke, focal and global ischemia, transient cerebral ischemic attacks or other cerebral vascular problems accompanied by cerebral ischemia. Other acute neurodegenerative conditions are associated with head trauma, spinal trauma, general anoxia, hypoxia, including fetal hypoxia, hypoglycemia, hypotension, as well as similar injuries seen during procedures from embole, hyperfusion or hypoxia.

The method of invention may be useful in a range of incidents, including during surgery, particularly cardiac surgery, in incidents of cranial hemmorhage, in perinatal asphyxia, in cardiac arrest, or status epilepticus, especially where blood flow to the brain is halted for a period of time.

The therapeutically effective amount provided in the treatment of a neurodegenerative disorder may vary according to the size, age and response pattern of the patient, the severity of the disorder, the judgment of the attending physician and the like. In general, effective amounts for daily oral administration may be about 0.01 to 1,000 mg/kg, preferably about 0.5 to 500 mg/kg and effective amounts for parenteral administration may be about 0.1 to 100 mg/kg, preferably about 0.5 to 50 mg/kg.

In actual practice, said 5-HT6 agonist is provided by administering the 5-HT6 agonist compound or a precursor thereof in a solid or liquid form, either neat or in combination with one or more conventional pharmaceutical carriers or excipients. Accordingly, the present invention provides a pharmaceutical composition for use in the treatment and prevention of a neurodegenerative disorder which comprises a pharmaceutically acceptable carrier and an effective amount of a 5-HT6 agonist as described hereinabove.

Solid carriers suitable for use in the composition of the invention include one or more substances which may also act as flavoring agents, lubricants, solubilizers, suspending agents, fillers, glidants, compression aides, binders, tablet-disintegrating agents or encapsulating materials. In powders, the carrier may be a finely divided solid which is in admixture with a finely divided 5-HT6 agonist compound. In tablets, said 5-HT6 agonist compound may be mixed with a carrier having the necessary compression properties in suitable proportions and compacted in the shape and size desired. Said powders and tablets may contain up to 99% by weight of the 5-HT6 agonist compound. Solid carriers suitable for use in the composition of the invention include calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Any pharmaceutically acceptable liquid carrier suitable for preparing solutions, suspensions, emulsions, syrups and elixirs may be employed in the composition of the invention. The 5-HT6 agonist compound may be dissolved or suspended in a pharmaceutically acceptable liquid carrier such as water, an organic solvent, or a pharmaceutically acceptable oil or fat, or a mixture thereof. Said liquid composition may contain other suitable pharmaceutical additives such as solubilizers, emulsifiers, buffers, preservatives, sweeteners, flavoring agents, suspending agents, thickening agents, coloring agents, viscosity regulators, stabilizers, osmo-regulators, or the like. Examples of liquid carriers suitable for oral and parenteral administration include water (particularly containing additives as above, e.g., cellulose derivatives, preferably sodium carboxymethyl cellulose solution), alcohols (including monohydric alcohols and polyhydric alcohols, e.g., glycols) or their derivatives, or oils (e.g., fractionated coconut oil and arachis oil). For parenteral administration the carrier may also be an oily ester such as ethyl oleate or isopropyl myristate.

Compositions of the invention which are sterile solutions or suspensions are suitable for intramuscular, intraperitoneal or subcutaneous injection. Sterile solutions may also be administered intravenously. Inventive compositions suitable for oral administration may be in either liquid or solid composition form.

For a more clear understanding, and in order to illustrate the invention more clearly, specific examples thereof are set forth hereinbelow. The following examples are merely illustrative and are not to be understood as limiting the scope and underlying principles of the invention in any way.

### EXAMPLE 1

Determination of the 5-HT6 Binding Affinity and cAMP Production of a Variety of 5-HT6 Ligands

A) Evaluation of 5-HT6 Binding Affinity of Test Compounds

The affinity of test compounds for the serotonin 5-HT6 receptor is evaluated in the following manner. Cultured Hela cells expressing human cloned 5-HT6 receptors are harvested and centrifuged at low speed (1,000×g) for 10.0 min to remove the culture media. The harvested cells are suspended in half volume of fresh physiological phosphate buffered saline solution and recentrifuged at the same speed. This operation is repeated. The collected cells are then homogenized in ten volumes of 50 mM Tris.HCl (pH 7.4) and 0.5 mM EDTA. The homogenate is centrifuged at 40,000×g for 30.0 min and the precipitate is collected. The obtained pellet is resuspended in 10 volumes of Tris.HCl buffer and recentrifuged at the same speed. The final pellet is suspended in a small volume of Tris.HCl buffer and the tissue protein content is determined in aliquots of 10-25 μl volumes. Bovine Serum Albumin is used as the standard in the protein determination according to the method described in Lowry et al., J. Biol. Chem., 193:265 (1951). The volume of the suspended cell membranes is adjusted to give a tissue protein concentration of 1.0 mg/ml of suspension. The prepared membrane suspension (10 times concentrated) is aliquoted in 1.0 ml volumes and stored at −70° C. until used in subsequent binding experiments.

Binding experiments are performed in a 96 well microtiter plate format, in a total volume of 200 μl. To each well is added the following mixture: 80.0 μl of incubation buffer made in 50 mM Tris.HCl buffer (pH 7.4) containing 10.0 mM MgCl₂ and 0.5 mM EDTA and 20 μl of [³H]-LSD (S.A., 86.0 Ci/mmol, available from Amersham Life Science), 3.0 nM. The dissociation constant, K_{D} of the [³H]LSD at the human serotonin 5-HT6 receptor is 2.9 nM, as determined by saturation binding with increasing concentrations of [³H]LSD. The reaction is initiated by the final addition of 100.0 μl of tissue suspension. Nonspecific binding is measured in the presence of 10.0 μM methiothepin. The test compounds are added in 20.0 μl volume.

The reaction is allowed to proceed in the dark for 120 min at room temperature, at which time, the bound ligand-receptor complex is filtered off on a 96 well unifilter with a Packard Filtermate® 196 Harvester. The bound complex caught on the filter disk is allowed to air dry and the radioactivity is measured in a Packard TopCount® equipped with six photomultiplier detectors, after the addition of 40.0 μl Microscint®-20 scintillant to each shallow well. The unifilter plate is heat-sealed and counted in a PackardTopCount® with a tritium efficiency of 31.0%.

Specific binding to the 5-HT6 receptor is defined as the total radioactivity bound less the amount bound in the presence of 10.0 μM unlabeled methiothepin. Binding in the presence of varying concentrations of test compound is expressed as a percentage of specific binding in the absence of test compound. The results are plotted as log % bound versus log concentration of test compound. Nonlinear regression analysis of data points with a computer assisted program Prism® yielded both the IC₅₀ and the Kᵢ values of test compounds with 95% confidence limits. A linear regression line of data points is plotted, from which the IC₅₀ value is determined and the Kᵢ value is determined based upon the following equation:
Kᵢ=IC₅₀/(1+L/K_{D})
where L is the concentration of the radioactive ligand used and K_{D} is the dissociation constant of the ligand for the receptor, both expressed in nM.

Using this assay, the following Ki values are determined and compared to those values obtained by representative compounds known to demonstrate binding to the 5-HT6 receptor. The data are shown in Table I below.

B) Determination of 5-HT6 Agonist Activity Using cAMP Accumulation

Intracellular cAMP levels are measured using 24-well plates containing the human 5-HT6 receptor stabily transfected into HELA cells. Upon initiation of the assay, the media from cell maintenance is aspirated and cells are preincubated at 37° C. for 15 mins. in KREBS buffer. Following this primary incubation, the buffer is aspirated and an additional incubation is performed at 37° C. for 5 mins. in KREBS buffer containing 500 uM IBMX (3-isobutyl-1-methylxanthine). Subsequently cells are incubated with test compound concentrations ranging from 10-6 to 10-11 M for 10 minutes at 37° C. The assay is terminated by the addition of 0.5M perchloric acid. Intracellular cAMP levels are determined by radioimmunoassay through the cAMP SPA screening kit. Data are analyzed graphically with GraphPad Prism (GraphPad Software, San Diego, Calif.). A 5-HT6 agonist is hereby defined as a compound which demonstrates ≧25% activity relative to the cAMP levels measured by the addition of serotonin (100 nM). The value is recorded as Emax (%) and shown on Table I.

TABLE I

| | 5-HT6 | % |
|---|---|---|
| Test Compound | Ki (nM) | Emax |
| A: (2-{3-[(2,5-dimethoxyphenyl)sulfonyl]-1H- | 3.6 | 95 |
| pyrrolo[2,3-b]pyridin-1-yl}ethyl)amine | | |
| B: N-(2-{3-[(3-fluorophenyl)sulfonyl]-1H-pyrrolo[2,3- | 5.0 | 100 |
| b]pyridin-1-yl}ethyl)-N,N-dimethylamine | | |
| C: 2-{1-[6-chloroimidazo[2,1-b][1,3]thiazol-5- | 2.0 | 93 |
| yl)sulfonyl]-1H-indol-3-yl}ethanamine | | |

### EXAMPLE 2

Evaluation of a 5-HT6 Agonist in Neuronal Survival

In this evaluation neuron cultures are prepared from E16 rat embryos. After a 24 h period, Test Compound B is added at various concentrations to the cultures. After a 72 h period, neuronal survival is determined by a neurofilament ELISA.

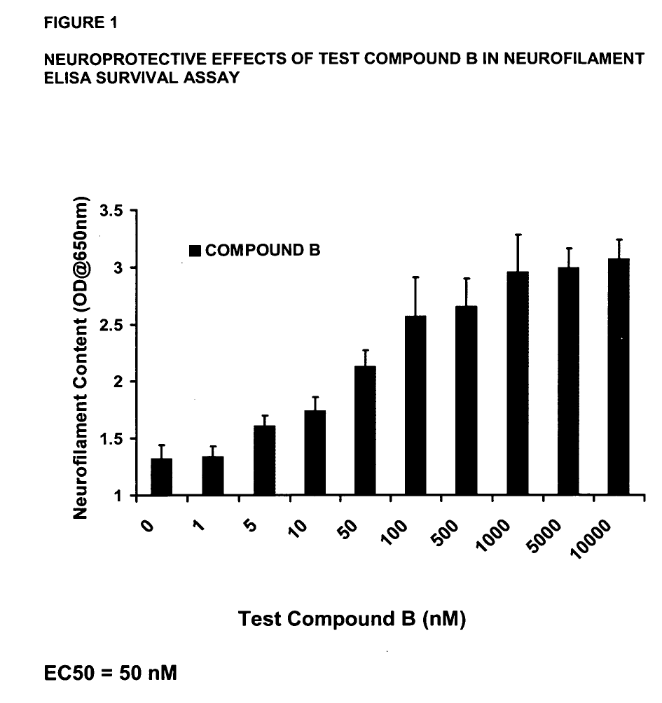

The data are expressed as total neurofilament content. The EC₅₀ for Test Compound B in this evaluation is 50 nM. The results are shown in FIG. 1

Results and Discussion:

As shown in FIG. 1, neuronal survival in culture is measured by the amount of neurofilament present after 72 h. Treatment of cultured neurons with Test Compound B increases the neurofilament content when compared to vehicle treated controls. The lowest concentration of Test Compound B providing significant enhancement of survival is 10 nM.

### EXAMPLE 3

Evaluation of a 5-HT6 Agonist on Neurite Outgrowth in Cultured Cortical Neurons

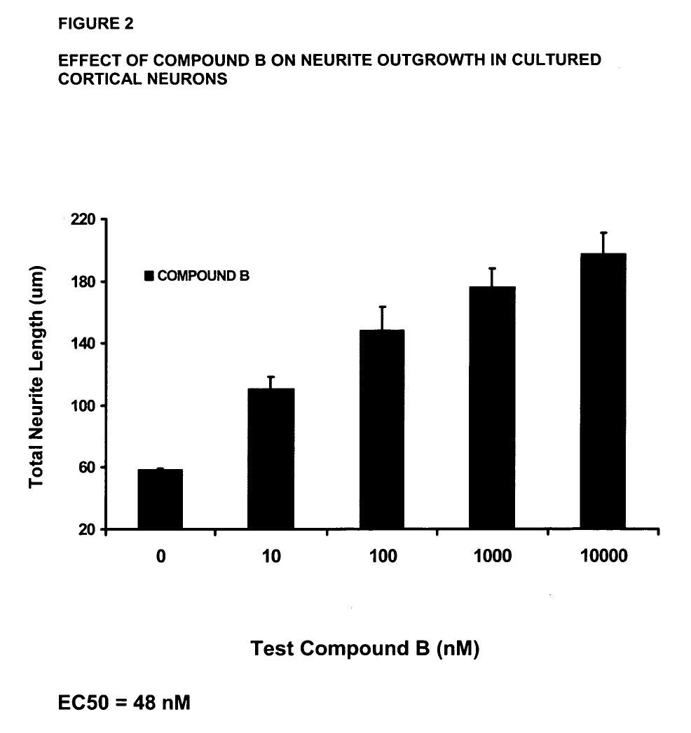

In this evaluation cortical neuron cultures are prepared from E16 rat embryos. After a 24 h period, Test Compound B is added at various concentrations to the cultures. Neurite outgrowth is determined after a 72 h period by staining cells with a tubulin antibody (TUJ-1) and measuring neurite length with the Cellomics ArrayScan, using the Enhanced Neurite Outgrowth (ENO) algorithm. The data are expressed as total neurite length. The EC₅₀ for Test Compound B in this evaluation is 48 nM. The results are shown in FIG. 2

Results and Discussion:

As shown in FIG. 2, total neurite length of cultured neurons is quantified after 72 h in culture. Treatment of cultured neurons with Test Compound B increased total neurite length when compared to vehicle treated controls. The lowest concentration of Test Compound B providing significant enhancement of total neurite length is 10 nM.

### EXAMPLE 4

Evaluation of the Neuroprotective Effect of a 5-HT6 Agonist Against Oxygen and Glucose Deprivation in Cerebellar Granule Neurons

Preparation of Cerebellar Granule neurons (CGN):

Cerebella isolated from P7 rat pup brains are cut into 1 mm pieces and transferred to a tube containing 0.3 mg/ml trypsin in HBSS. Following enzymatic digestion, the tissue is mechanically triturated. The supernatant is collected and centrifuged at 1200 rpm for 10 minutes. The resultant pellet is resuspended in complete media (Neurobasal, 25 nM potassium, 0.5 mM L-glutamine, 100 U/ml penicillin, 100 μg/ml streptomycin, 10% FBS) and plated at a density of 0.5×10⁶ cells/well in 24-well plates. After 24 h, media is exchanged with complete serum-free medial (Neurobasal, 25 nM potassium, 0.5 mM L-glutamine, 100 U/ml penicillin, 100 μg/ml streptomycin, B-27 supplement).

Oxygen Glucose Deprivation (OGD) in CGN:

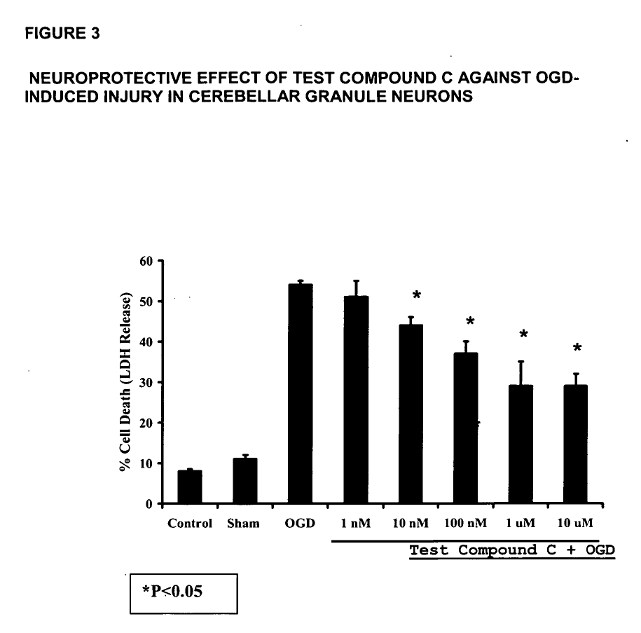

This is a well characterized model of ischemia-like neuronal injury involving glutamate-induced excitotoxicity (A. Kaasik, et al, Neuroscience (2001) 102, pp427-432). Cultures are maintained for 14 days in vitro prior to experimentation. Cultures are pretreated for 1 h with various concentrations of Test Compound C and transferred to an anaerobic chamber, where the media is exchanged with deoxygenated buffer and maintained for 4 h in the presence of fresh Test Compound C. Following the 4 h OGD, deoxygenated buffer is exchanged with complete media. Cultures are maintained for 24 h in the presence of fresh Test Compound C. At the end of the 24-h period, cell death is determined by measuring lactate dehydrogenase (LDH) released into the media and is expressed as a percent of cell death. The results are shown in FIG. 3 and are presented as means±SD from 5 experiments. Sham bar represents results obtained with cultures subjected to manipulations and changes of culture media without OGD or drug treatment.

Results and Discussion:

As shown in FIG. 3, exposure of CGN cultures to 4 h of OGD results in over 50% of cell death as measured by a release of a cytoplasmic enzyme (LDH) into the culture medium. Pretreatment of cultured neurons with Test Compound C, followed by their exposure to OGD and a 24 h recovery period in the presence of Test Compound C results in a dose-dependent reduction of neuronal cell death. The lowest concentration of Test Compound C providing significant protection is 10 nM. Treatment with 1 μM Test Compound C reduced neuronal death by 50%.

### EXAMPLE 5

Evaluation of the Neuroprotective Effect of a 5-HT6 Agonist Against Potassium Withdrawal-Induced Apoptosis in Cerebellar Granule Neurons

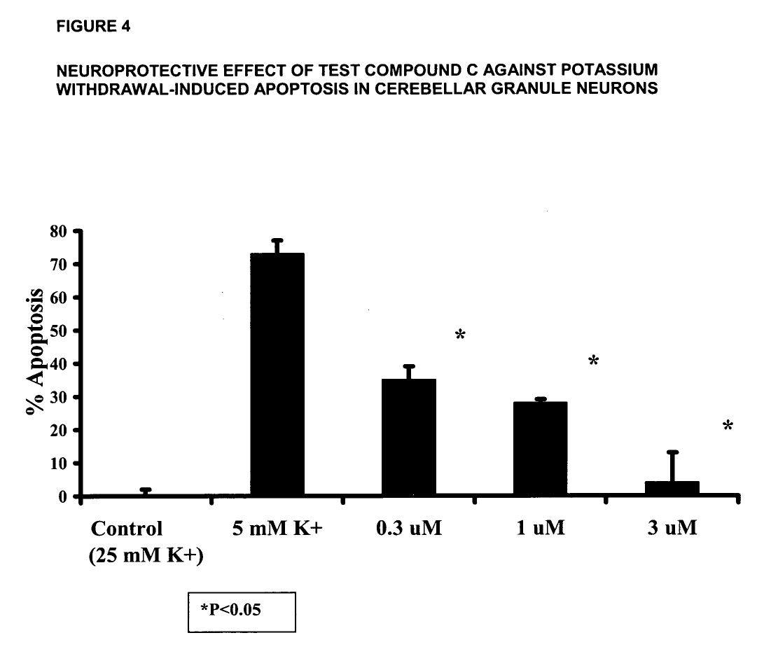

Potassium withdrawal, i.e. replacement of 25 nM potassium in the CGN culture medium is a well established model of neuronal cell death by apoptosis (T. M. Miller and E. M. Johnson, Journal of Neuroscience, (1996) 16(23), pp7487-7495). Cultures are maintained for 7 days prior to experimentation. Complete media containing 25 mM K⁺ is exchanged with complete media containing 5 mM K⁺. Various concentrations of Test Compound C are added to the cultures. After 24 h, apoptotic cell death is determined by measuring DNA fragmentation via ELISA. Apoptotic cell death is expressed as % apoptosis. The results are presented as means±SD from two experiments and are shown in FIG. 4.

Results and Discussion:

As can be seen in FIG. 4, a replacement of 25 mM potassium with 5 mM potassium induces 75% apoptotic neuronal cell death after a 24 h interval. The presence of Test Compound C during the low potassium treatment significantly, and dose dependently, attenuated the amount of fragmented DNA used as a measure of apootosis in this model. The lowest concentration of Test Compound C evaluated (0.3 μM) reduced cell death by 50% while almost total protection from apoptosis was found in the presence of 3.0 μM of Test Compound C.

### EXAMPLE 6

Evaluation of the Effect of a 5-HT6 Agonist on Brain Derived Neurotrophic Factor Protein Levels in Cultured Cortical Neurons

In this evaluation cortical neuron cultures are prepared from E16 rat embryos and plated on pre-coated 10 cm Petri dishes. After a 24 h period, Test Compound B is added to the cultures. Levels of brain derived neurotrophic factor (BDNF) protein were measured after a 72 h period by lysing the cells and using a BDNF sandwich ELISA. More specifically, ELISA plates were coated with an anti-BDNF monoclonal antibody. Nonspecific binding was blocked and 50 μg of sample protein was added. A second anti-BDNF antibody was added and incubated. An anti-IgY antibody conjugated with horseradish peroxidase was added and incubated. A TMB solution was added and the colorimetric reaction was measured in a plate reader (absorbance of 450 nm). BDNF levels are quantified after 72 h in culture. The data are graphically shown in FIG. 6.

Results and Discussion:

As shown in FIG. 6, treatment of cultured neurons with Test Compound B significantly increased BDNF protein levels when compared to vehicle treated control.

What is claimed is:

1. A method for the treatment of a neurodegenerative disorder in a patient in need thereof which comprises providing to said patient a therapeutically effective amount of a 5-hydroxytryptamine-6 agonist.

2. The method according to claim 1 wherein the 5-hydroxytryptamine-6 agonist is a 1-sulfonyltryptamine derivative.

3. The method according to claim 1 wherein the 5-hydroxytryptamine-6 agonist is a 1-aminoalkyl-3-sulfonylazaindole derivative.

4. The method according to claim 1 wherein the 5-hydroxytryptamine-6 agonist is a compound of formula I 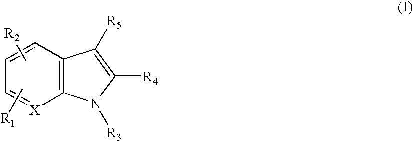
wherein
X is CH or N;
R₁ and R₂ are each independently H, halogen, CN, OCO₂R₁₂, CO₂R₁₃, CONR₁₄R₁₅, CNR₁₆NR₁₇R₁₈, SOₘR₁₉, NR₂₀R₂₁, OR₂₂, COR₂₃ or a C₁-C₆alkyl, C₂-C₆alkenyl, C₂-C₆alkynyl, C₃-C₆cycloalkyl, cycloheteroalkyl, aryl or heteroaryl group each optionally substituted;
R₃ is SO₂R₈ when X is CH or (CH₂)ₙNR₆R₇ when X is N;
R₄ is H, halogen, or a C₁-C₆alkyl, C₁-C₆alkoxy, aryl or heteroaryl group each optionally substituted;
R₅ is (CH₂)ₙNR₆R₇ when X is CH or SO₂R₈ when X is N;
n is an integer of 2 or 3;
R₆ and R₇ are each independently H or a C₁-C₆alkyl, C₂-C₆alkenyl, C₂-C₆alkynyl, C₃-C₆cycloalkyl, cycloheteroalkyl, aryl or heteroaryl group each optionally substituted, or R₆ and R₇ may be taken together with the atom to which they are attached to form an optionally substituted 5- to 7-membered ring optionally containing an additional heteroatom selected from O, N or S;
R₈ is an optionally substituted aryl, heteroaryl or 8- to 13-membered bicyclic or tricyclic ring system having a N atom at the bridgehead and optionally containing 1, 2 or 3 additional heteroatoms selected from N, O or S;
m is 0 or an integer of 1 or 2;
R₁₂, R₁₃, R₁₉ and R₂₃ are each independently H or a C₁-C₆alkyl, C₂-C₆alkenyl, C₂-C₆alkynyl, C₃-C₆cycloalkyl, cycloheteroalkyl, aryl or heteroaryl group each optionally substituted;
R₁₄, R₁₅ and R₂₂ are each independently H or an optionally substituted C₁-C₆alkyl group; and
R₁₆, R₁₇, R₁₈, R₂₀ and R₂₁ are each independently H or an optionally substituted C₁-C₄alkyl group; or R₂₀ and R₂₁ may be taken together with the atom to which they are attached to form a 5- to 7-membered ring optionally containing another heteroatom selected from O, N or S; or
a stereoisomer thereof or a pharmaceutically acceptable salt thereof.

5. The method according to claim 4 having the formula I compound wherein X is CH; n is 2; and R₈ is a phenyl or imidazo[2,1-b][1,3]thiazolyl group each optionally substituted.

6. The method according to claim 4 having a formula I compound wherein X is N; n is 2; and R₈ is a phenyl or imidazo[2,1-b][1,3]thiazolyl group each optionally substituted.

7. The method according to claim 4 having a formula I compound selected from the group consisting of:
2-{1-[6-chloroimidazo[2,1-b][1,3]thiazol-5-yl)sulfonyl]-1H-indol-3-yl}ethanamine;
(2-{3-[(2,5-dimethoxyphenyl)sulfonyl]-1H-pyrrolo[2,3-b]pyridin-1-yl}ethyl)amine;
N-(2-{3-[(3-fluorophenyl)sulfonyl]-1H-pyrrolo[2,3-b]pyridin-1-yl}ethyl)-N,N-dimethylamine;
2-{[1-(phenylsulfonyl)-1H-indol-3-yl]ethyl}-N,N-dimethylamine;
a pharmaceutically acceptable salt thereof; and
a stereoisomer thereof.

8. The method according to claim 1 wherein said disorder is an acute neurodegenerative disorder.

9. The method according to claim 1 wherein said disorder is a chronic neurodegenerative disorder.

10. The method according to claim 8 wherein said disorder is selected from the group consisting of stroke; head trauma; spinal trauma; and asphyxia.

11. The method according to claim 9 wherein said disorder is selected from the group consisting of Alzheimer's disease; Huntington's disease; Parkinson's disease; epilepsy; amyotrophic lateral sclerosis; AIDS dementia; and retinal disease.

12. The method according to claim 4 wherein said disorder is stroke or Alzheimer's disease.

13. A pharmaceutical composition which comprises a pharmaceutically acceptable carrier and an effective amount of a 5-HT6 agonist.

14. The composition according to claim 13 wherein said 5-HT6 agonist is a compound of formula I 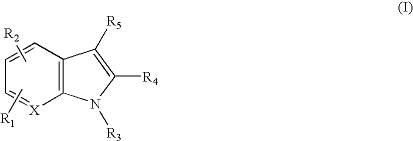
wherein
X is CH or N;
R₁ and R₂ are each independently H, halogen, CN, OCO₂R₁₂, CO₂R₁₃, CONR₁₄R₁₅, CNR₁₆NR₁₇R₁₈, SOₘR₁₉, NR₂₀R₂₁, OR₂₂, COR₂₃ or a C₁-C₆alkyl, C₂-C₆alkenyl, C₂-C₆alkynyl, C₃-C₆cycloalkyl, cycloheteroalkyl, aryl or heteroaryl group each optionally substituted;
R₃ is SO₂R₈ when X is CH or (CH₂)ₙNR₆R₇ when X is N;
R₄ is H, halogen, or a C₁-C₆alkyl, C₁-C₆alkoxy, aryl or heteroaryl group each optionally substituted;
R₅ is (CH₂)ₙNR₆R₇ when X is CH or SO₂R₈ when X is N;
n is an integer of 2 or 3;
R₆ and R₇ are each independently H or a C₁-C₆alkyl, C₂-C₆alkenyl, C₂-C₆alkynyl, C₃-C₆cycloalkyl, cycloheteroalkyl, aryl or heteroaryl group each optionally substituted, or R₆ and R₇ may be taken together with the atom to which they are attached to form an optionally substituted 5- to 7-membered ring optionally containing an additional heteroatom selected from O, N or S;
R₈ is an optionally substituted aryl, heteroaryl or 8- to 13-membered bicyclic or tricyclic ring system having a N atom at the bridgehead and optionally containing 1, 2 or 3 additional heteroatoms selected from N, O or S;
m is 0 or an integer of 1 or 2;
R₁₂, R₁₃, R₁₉ and R₂₃ are each independently H or a C₁-C₆alkyl, C₂-C₆alkenyl, C₂-C₆alkynyl, C₃-C₆cycloalkyl, cycloheteroalkyl, aryl or heteroaryl group each optionally substituted;
R₁₄, R₁₅ and R₂₂ are each independently H or an optionally substituted C₁-C₆alkyl group; and
R₁₆, R₁₇, R₁₈, R₂₀ and R₂, are each independently H or an optionally substituted C₁-C₄alkyl group; or R₂₀ and R₂₁ may be taken together with the atom to which they are attached to form a 5- to 7-membered ring optionally containing another heteroatom selected from O, N or S; or
a stereoisomer thereof or a pharmaceutically acceptable salt thereof.

15. The composition according to claim 14 having the formula I compound wherein X is CH; n is 2; and R₈ is a phenyl or imidazo[2,1-b][1,3]thiazolyl group each optionally substituted.

16. The method according to claim 14 having a formula I compound wherein X is N; n is 2; and R₈ is a phenyl or imidazo[2,1-b][1,3]thiazolyl group each optionally substituted.

17. The method according to claim 14 having a formula I compound selected from the group consisting of:
2-{1-[6-chloroimidazo[2,1-b][1,3]thiazol-5-yl)sulfonyl]-1H-indol-3-yl}ethanamine;
(2-{3-[(2,5-dimethoxyphenyl)sulfonyl]-1H-pyrrolo[2,3-b]pyridin-1-yl}ethyl)amine;
N-(2-{3-[(3-fluorophenyl)sulfonyl]-1H-pyrrolo[2,3-b]pyridin-1-yl}ethyl)-N,N-dimethylamine;
2-{[1-(phenylsulfonyl)-1H-indol-3-yl]ethyl}-N,N-dimethylamine;
a pharmaceutically acceptable salt thereof; and
a stereoisomer thereof.
